(19) United States
(12) Patent Application Publication
(10) Pub. No.: US 2026/0345974 A1
(43) Pub. Date: Nov. 5, 2026

(54) EXTERNAL VENTRICULAR DRAIN ALIGNMENT

(71) Applicant: Worcester Polytechnic Institute, Worcester, MA (US)

(72) Inventors: Rohit Dey, Worcester, MA (US); Yihao Zheng, Westwood, MA (US)

(21) Appl. No.: 19/642,995

(22) Filed: Apr. 9, 2026

Related U.S. Application Data
(60) Provisional application No. 63/786,176, filed on Apr. 9, 2025.

(51) Int. Cl. A61M 27/00

(52) U.S. Cl. CPC A61M 27/006; A61M 2027/004; A61M 2202/0014; A61M 2202/0464; A61M 2205/3344; A61M 2205/82

(57) ABSTRACT

An automated pressure sensing and feedback control for adjusting the elevation of an external ventricular drain (EVD) for maintaining a constant, therapeutically beneficial pressure in the EVD. In contrast to conventional EVDs, which require nurses to frequently adjust settings—sometimes multiple times per hour—the disclosed approach automatically maintains pre-set pressure difference in the EVD, regardless of patient movement. Measurement of fluid in separate vessels based upon a reference, equilibrium level detects a pressure difference when a measured fluid vessel has a pressure difference with the reference fluid vessel resulting from patient movement or elevation. Height adjustment of the EVD to adjust the vertical displacement again achieves equilibrium indicating realignment of the EVD with the patient.

### RELATED APPLICATIONS

This patent application claims the benefit under 35 U.S.C. § 119(e) of U.S. Provisional Patent App. No. 63/786,176, filed Apr. 9, 2025, entitled “EXTERNAL VENTRICULAR DRAIN ALIGNMENT,” incorporated herein by reference in entirety.

### BACKGROUND

Surgical drains are often employed for relief of excess fluids generated by normal physiological processes in response to surgical procedures, typically as a healing response to the tissue regions affected by the surgery. Many drains simply pursue a downward flow to a collection bag or bandage, however some regions are more sensitive. In the case of intercranial procedures, for example, cerebrospinal fluid (CSF) is preferable to maintain at a volume and pressure conducive to healing, to avoid compressing sensitive brain tissue from excess fluid or creating voids prone to infection from excessive fluidic evacuation.

### SUMMARY

Configurations herein are based, in part, on the observation that surgical fluid drains such as external ventricular drains (EVDs) for cerebrospinal fluid (CSF) are often employed for collecting and evacuating fluid from a surgical region for mitigating an excessive accumulation of fluid that can increase pressure and infection risks in the surgical region. In the case of patient recovery employing EVDs from cranial surgical procedures, precise and consistent management of intracranial pressure (ICP) from CSF accumulation is critical. Patient movement, such as alternating between lying, elevated, and sitting positions affects the pressure in the EVD because of head elevation. Unfortunately, conventional approaches to EVD management rely on manual adjustments to maintain the appropriate ICP level, which can be time-consuming, prone to human error, and disruptive for patients. This issue is especially pressing in critical care settings, where patients may frequently be repositioned or experience sudden changes in ICP.

Accordingly, configurations herein substantially overcome the shortcomings of manual height adjustments for EVD pressure mitigation by providing an automated pressure sensing and feedback control for adjusting the elevation of the EVD for maintaining a constant, therapeutically beneficial pressure in the EVD. In contrast to conventional EVDs, which require nurses to frequently adjust settings—sometimes multiple times per hour—the disclosed approach automatically maintains pre-set pressure difference in the EVD, regardless of patient movement. It is specifically designed to counteract any pressure fluctuations caused by movement or repositioning, ensuring consistent ICP management while minimizing disruptions for the patient. This not only saves valuable nursing time but also reduces the risk of complications. While some conventional approaches purport to manage ICP, the disclosed approach is distinguished by being completely isolated from cerebrospinal fluid (CSF), which helps prevent contamination and infections.

In further detail, a device for maintaining pressure in a surgical drain includes a reference to a fluid pressure based on a fluid level resulting from a physiological process such as post-surgical cerebrospinal fluid, and a sensor responsive to a pressure change based on a vertical displacement from the reference. A receptacle connected to a linear actuator is adapted to engage a fluid management drain configured for receiving excess fluid generated by the physiological process based on a pressure defined by the fluid level. A control circuit computes the vertical displacement based on pressure signals from the sensor, such that the linear actuator is responsive to the sensor for correcting the vertical displacement relative to the reference.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a surgical recovery environment suitable for use with fluidic collection systems and devices as disclosed herein;

FIG. 2 shows a surgical fluid drain pressure management device disposed in the environment of FIG. 1;

FIGS. 3A and 3B show views of a patient engagement with the drain pressure management device consistent with configurations herein;

FIGS. 4A-4C show the drain pressure management device engaged with an external ventricular drain for fluid management;

FIGS. 5A-5C show the drain pressure management device as in FIGS. 3A-4C installed on a therapeutic equipment stand;

FIG. 6 shows a process flow of linear actuator control based on the control circuit in the drain pressure management device;

FIG. 7 shows a circuit schematic of memory and processor architecture in the control circuit of FIG. 6; and

FIG. 8 show computations of fluid mechanics as performed by the control circuit of FIG. 7.

### DETAILED DESCRIPTION

The disclosed surgical drain pressure management device seamlessly integrates with current External Ventricular Drainage (EVD) systems and automatically adjusts its level to maintain a pre-set constant pressure difference, invariant to the patient's movement.

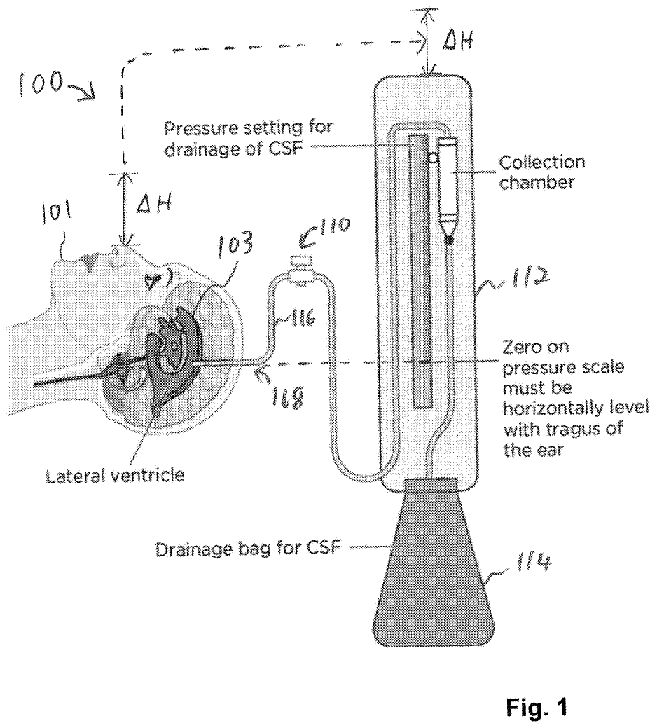

FIG. 1 is a context diagram of a surgical recovery environment 100 suitable for use with fluidic collection systems and devices as disclosed herein. The pressure management device automatically maintains the pre-set pressure difference, regardless of patient movement. It is specifically designed to counteract any pressure fluctuations caused by movement or repositioning, ensuring consistent ICP management while minimizing disruptions for the patient. This not only saves valuable nursing time but also reduces the risk of complications. Although some automated EVDs are available, The disclosed approach is distinguished by being completely isolated from cerebrospinal fluid (CSF), which helps prevent contamination/infections. In medical terms, the cerebrospinal fluid is a clear, colorless liquid circulating in the ventricles of the brain, the subarachnoid space, and the spinal cord central canal. It cushions the central nervous system, provides nutrients, removes waste, and maintains chemical stability.

Referring to FIG. 1, in a surgical recovery environment 100, a patient 101 may receive a surgical drain 110 including an EVD collector device (EVD) 112, a collection container 114, and a fluid vessel 116 in communication with a lateral ventricle 103 or other surgical access for receiving fluid accumulation. The position of the vessel 116 provides a reference 118 to a fluid pressure based on a fluid level resulting from a physiological process such as the CSF production. The disclosed device for maintaining pressure in the surgical drain vessel 116 relies on gravitationally induced fluid mechanics and maintaining a consistent height of the EVD 112 with the reference 118. Change to the height λH brought about by patient movement (e.g. lying, prone, sitting) are offset by a corresponding change in the height of the EVD 112 for maintaining a consistent pressure. While the disclosed fluid level is based on a patient cerebrospinal fluid (CSF) accumulation, alternate approaches may address other pressure sensitive accumulations of excess fluid accumulations.

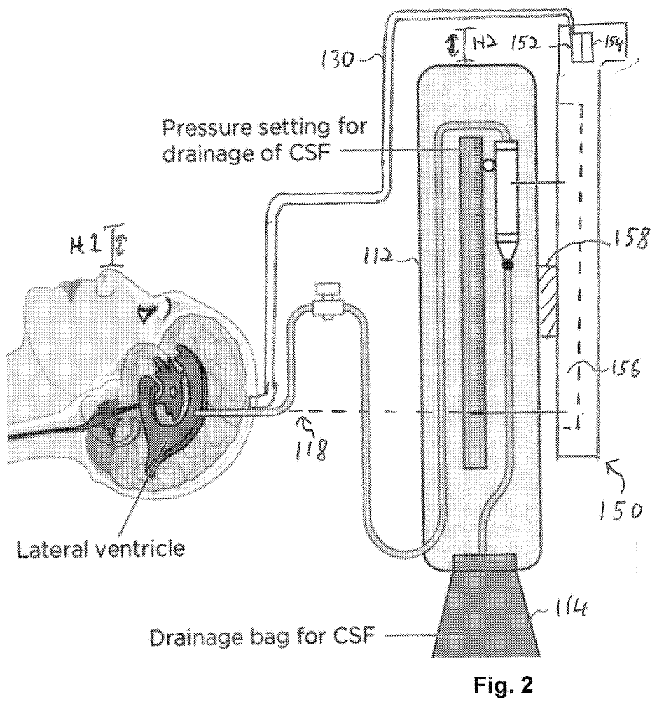

FIG. 2 shows a surgical fluid disposed in the environment of FIG. 1. Referring to FIGS. 1 and 2, the pressure management device 150 includes a sensor 152 responsive to a pressure change based on a vertical displacement from the reference 118, and a linear actuator 156. A receptacle 158 connects to the linear actuator 156 and is adapted to engage a fluid management drain such as the EVD 112 configured for receiving excess fluid generated by the physiological process based on a pressure defined by the fluid level. Based on a detected change in vertical elevation H1, the linear actuator 156 is responsive to the sensor 152 for correcting the vertical displacement relative to the reference 118 to change H2 equal to H1.

In the configurations herein, a circuit 154 computes the vertical displacement based on pressure signals from the sensor 152. At least one fluid vessel 130 is coupled to the location of the reference 118, such that the sensor 152 in communication with the fluid vessel 130 and is configured for detecting a pressure change therein. The vertical displacement H1 is generally defined by a height difference from the fluid level to the reference, meaning an elevation of the patient 101, while lateral movement (as in forward movement to a sitting position) need not be detected since the elevation or height is the primary driver of the pressure.

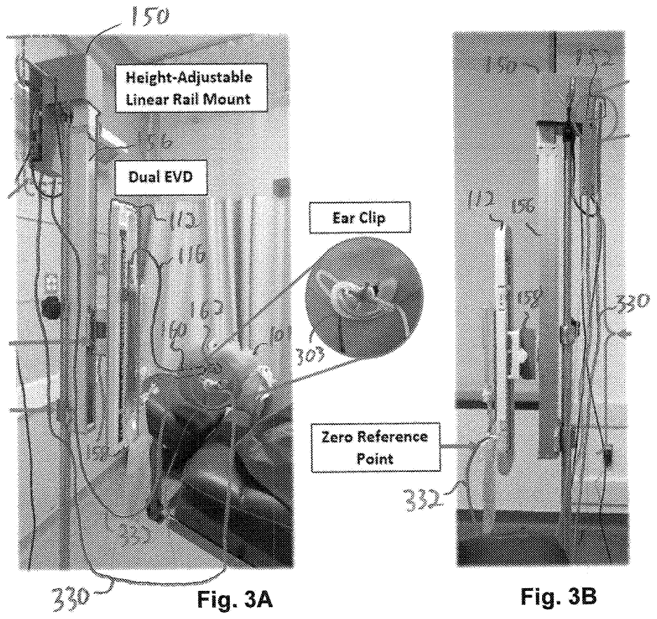

FIGS. 3A and 3B show views of a patient engagement with the drain pressure management device consistent with configurations herein. FIG. 3A shows a perspective front view of the patient 101 and EVD 112 engaged with the pressure management device 150. In a patient 101, the CSF drain typically takes the form of a cannula 160 inserted from a suitable position, typically the lower rear skull, and includes an array of fenestrations 162 to allow drainage of the CSF.

In the configuration of FIG. 3, rather than a single fluid vessel 130, a measured fluid vessel 330 and a reference fluid vessel 332 are invoked, such that the sensor 152 is configured to detect an inequality in pressure between the measured fluid vessel 330 and the reference fluid vessel 332. The measurement circuit 154 is connected to the sensor 152, such that the sensor receives a measured pressure from the measured fluid vessel 330 and a reference pressure from the reference fluid vessel 332.

In the configuration of FIG. 3A, the measured fluid vessel 330 attaches to an ear clip 303, which in typical procedures defines the reference 118 of the CSF drain, as the lateral ventricle is located between the ears. The reference fluid vessel 332 connects to a zero reference location 318 based on the EVD. When the height of the EVD 112 is such that the zero reference location 318 is aligned (level) with the ventricle cannula 160, appropriate pressure is established. With the reference fluid vessel 332 contains fluid meeting the zero reference location 318, and the fluid in the measured fluid vessel 330 meeting the ear, and thus matching the CSF level, sensed pressure in both vessels 330, 332 is equal.

The linear actuator 156 is configured for adjusting a height of the fluid management drain 112 for attaining an equality of the reference pressure and the measured pressure. By employing a differential pressure sensor as the sensor 152, the circuit 154 is configured to energize the linear actuator 156 until a pressure equality is detected in response to the vertical displacement. The vertical movement of the patient's head defines the vertical displacement and corresponding pressure change in the measured fluid vessel 330. The linear actuator 156 moves the EVD 112 up or down until the pressure is again equal, indicating realignment of the EVD zero reference location 118 with the CSF cannula 160, as defined by the ear clip 303.

The configuration provides a device and method to automatically align the zero-pressure datum to the patient's tragus, ensuring accurate CSF drainage during movement, while integrating with existing EVD setups and maintaining standard operations. The tragus, beneficially, is a small, cartilaginous projection or flap of skin-covered tissue located immediately in front of the external auditory canal (ear opening) and behind the face. It is a prominent, firm structure on the outer ear that provides an accurate location of the CSF containing lateral ventricle upon which the drain is referenced.

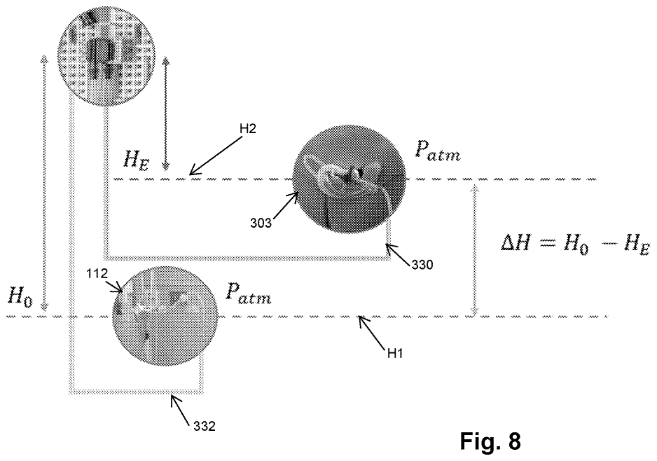

FIG. 3B shows a side elevation with the measured fluid vessel 330 and the reference fluid vessel 332 connected to the differential pressure sensor 152. The fluid vessels 330, 332 therefore depict fluid mechanics of a fluid column. The differential sensor 152 senses the pressure difference induced by the continuous fluid columns attached to EVD's zero-reference point and the patient's ear using the differential pressure sensor 152 or transducer. This difference in the pressure between the EVD's zero reference point and patients'ear (targus) is directly proportional to the height difference in accordance to Pascal's law of transmission of fluid-pressure, discussed further below in FIG. 8. The disclosed device 150 uses this information to actively control the linear actuator 156 to adjust the height of the EVD 112 to maintain a constant set pressure difference whenever the pressure deviates from the set-reference. This technique is robust and can achieve sub-millimeter-level accuracy, providing precise and reliable ICP management.

In contrast to conventional approaches, such as that disclosed in U.S. Pat. No. 9,717,890, the sensor 152 does not require fluid communication with the CSF. Exposure or contact with the CSF increases a risk of infection and contamination. Other conventional approaches, such as that disclosed in U.S. Patent Pub. No. 2021/0060315,suggests a single vessel and computed pressure values by collecting fluid in a gravity fed vessel below the CSF reference and EVD. The '315 publication also requires a complex elevated post for pressure correction, rather than integrating with a standard IV post for suspending the EVD, discussed further below.

A further diagnostic and corrective actuation can provide gentle agitation of the CSF fluid for dislodging sediment or blockage from accumulating in the cannula 160. The CSF fluid level emanates from the intercranial cannula 160 having fenestrations 162 for CSF flow. The fenestrations 162 can be small and prone to stagnation from a sleeping or stationary patient. In a remedial cycle, the circuit 152 is configured for an agitation of the fluid from the physiological process by a cyclic variation of a vertical position of the fluid management drain for iterative fluid level variation. Modest (less than 1 or 2 cm) in elevation can generate subtle fluid currents that are not detrimental to the patient 101, but may disrupt settlement or stagnant fluid to prevent blockage.

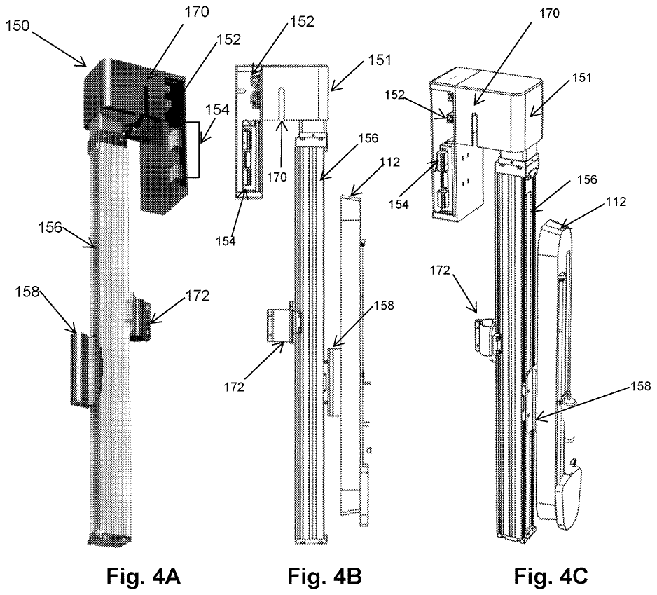

FIGS. 4A-4C show the drain pressure management device engaged with the EVD for fluid management. Referring to FIGS. 1 and 4A-4C, the drain pressure management device 150 is a self contained interface between a mounting structure, such as an therapeutic equipment stand or IV stand, and includes a hanging slot 170 and a clamp 172 for engaging a vertical upright post of the IV stand. The EVD device 112, typically also adapted to engage an IV stand, attaches to a receptacle 158 or protrusion that emulates the vertical upright. The receptacle 158 extends from the linear actuator 156 which controls the height for accommodating vertical displacement. A housing 151 contains the control circuit 154, typically on a circuit board, and the sensor 152, along with support structures for the linear actuator 156 and hanging slot 170.

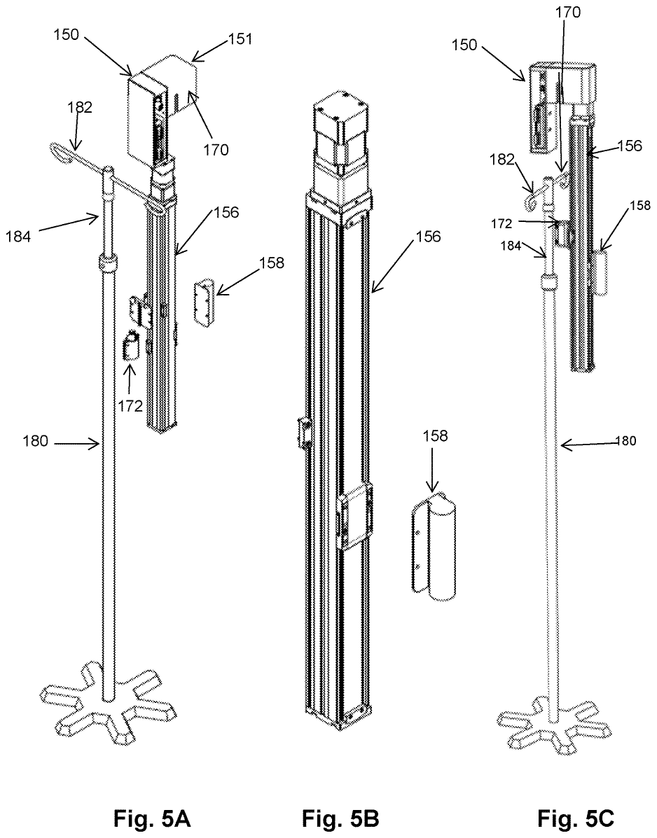

FIGS. 5A-5C show the drain pressure management device as in FIGS. 3A-4C installed on a therapeutic equipment stand 180. Referring to FIGS. 1 and 3A-5C, an exploded view in FIG. 5A shows the hanging slot 170 of the housing 151 adapted to engage a horizontal rod 182 of the equipment stand 180. The housing 151 suspends the linear actuator 156 via the slot 170 and clamp 172 around a vertical upright 184 of the stand 180, which in turn suspends the EVD deice 112 via the receptacle 158. Collectively, the hanging slot 170 and clamp 172 provide the attachment to the therapeutic equipment stand 180 for suspending the linear actuator and the receptacle at an elevation above the reference 118, such that the elevation induces the pressure change based on gravitational forces on the fluid vessels 130, 330, 332. In contrast to conventional approaches, such as that disclosed in the '315 publication cited above, the device 150 engages with a standard IV hanging stand, without a need to drive or elevate the stand because it interfaces between the unmodified stand and the EVD device 112.

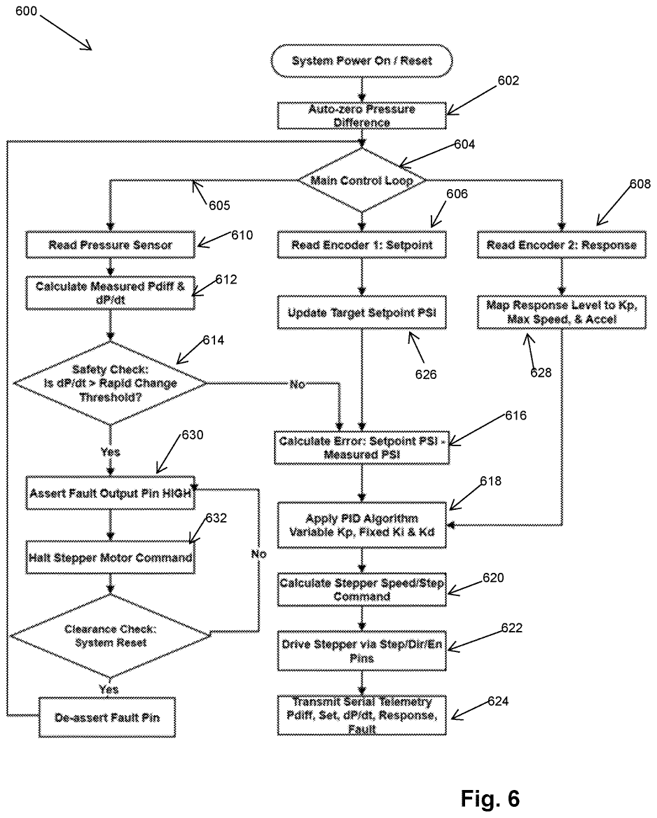

FIG. 6 shows a process flow 600 of linear actuator control based on the control circuit in the drain pressure management device. In operation, the circuit 154 receives signals from the sensor 152 to iteratively monitor the height H1 and adjust H2 accordingly, if needed. An auto zero step 602 defines a zero or equilibrium reference in the sensor 152, presumably when the EVD 112 aligns with the reference 118. A main control loop 604 periodically examines on-board encoders at step 606 and 608, and otherwise commences a main control path 605 by reading the pressure sensor 152 at step 610. A difference from previous readings is computed at step 612, and a safety check is performed at step 614 for rapid or extreme pressure changes.

The CSF pressure is expected within a certain range based on normal patient movement. A detached ear clip or ruptured vessel, such as from an ear clip falling on the floor, would generate an erroneous reading beyond a safety threshold. The check at step 614 prevents extreme actuator 156 movement based on error conditions which could otherwise cause uncomfortable or dangerous pressure management by the actuator 156.

If the check at step 614 is within a normal threshold, a difference is calculated at step 616. A new position is computed at step 618, typically by proportional differential (P/D) controller logic, and stepper motor commands computed at step 620 for directing the linear actuator 156. A corresponding control signal is sent at step 622 to advance the liner actuator 156 to the new position defined by H2, and any faults evaluated at step 624.

If encoder inputs are detected, at step 626 a target setpoint, typically an adjustment to the height based on a zero difference/equality at the reference 118, may be applied. Also, adjustment to motor defaults may be applied at step 628. If a fault is detected at step 614, a fault signal is asserted, as depicted at step 630 and the movement of the liner actuator 156 halted, as shown at step 632 until the system is reevaluated and the fault cleared.

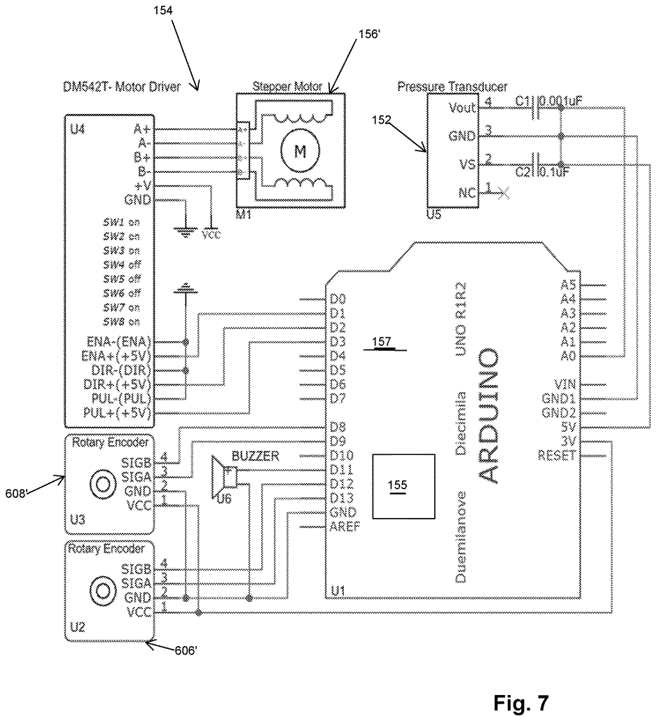

FIG. 7 shows a circuit schematic of memory and processor architecture in the control circuit of FIG. 6. The circuit 154 includes a processor 157 and a memory 155, such that the memory includes instructions for the control loop (FIG. 6) for computing the height differential, where the linear actuator 156 is responsive to the control loop based on the computed height differential. A stepper motor 156′ drives the linear actuator 156 as directed by the processor 157. Rotary encoders 606′ and 608′ correspond to the encoder reading steps 606 and 608, respectively, in FIG. 6. The sensor 152, in a typical configuration, is defined by a differential pressure transducer connected to the processor 157.

FIG. 8 show computations of fluid mechanics as performed by the control circuit of FIG. 7. Referring to FIGS. 1-8, the method for maintaining cerebrospinal fluid (CSF) levels in a CSF drain of an intercranial surgical patient 101 includes locating the reference fluid vessel 332 in association with a vertical location H1 defined by an external ventricular drains (EVD) device 112 for maintaining a pressure in the CSF drain 116. The H2 readings is evaluated from the measured fluid vessel 330 in association with a patient drain position defined by a CSF drain cannula 160. The vessels 330, 332 connect to the drain pressure management device 150, which suspended from the therapeutic equipment stand 180, and engages the external ventricular drains (EVD) device 112 via the linear actuator for vertical adjustment, typically mounted above the reference 118 EVD drain location. Using pressure readings from the vessels 330, 332, the working principle is shown by Pascal's law of hydrostatic pressure:

ΔPₜ=ρg·Δh

To sense displacement of the measured fluid vessel 330 and corresponding height difference, it is recognized that a pressure exerted by a liquid column is only dependent on the height of the liquid column. From this, the circuit 152 applies the proportional differential (PD) Control:

Motor Speed(t)=kᵢ*ΔPₜ+k_{d}*(ΔPₜ−ΔPₜ₋₁)

for activating the linear actuator 156 from the control circuit 152 for maintaining a vertical alignment of the EVD device 112 with the CSF drain cannula 160/reference 118 based on a pressure equality between the reference fluid vessel 332 and the measured fluid vessel 330. Following actuation, the EVD device 112 is realigned with the CSF drain cannula based on a detecting a subsequent a pressure equality between the reference fluid vessel 332 and the measured fluid vessel 330.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A device for maintaining pressure in a surgical drain, comprising:
a reference to a fluid pressure based on a fluid level resulting from a physiological process;
a sensor responsive to a pressure change based on a vertical displacement from the reference;
a linear actuator; and
a receptacle connected to the linear actuator and adapted to engage a fluid management drain configured for receiving excess fluid generated by the physiological process based on a pressure defined by the fluid level,
the linear actuator responsive to the sensor for correcting the vertical displacement relative to the reference.

2. The device of claim 1 further comprising a circuit for computing the vertical displacement based on pressure signals from the sensor.

3. The device of claim 1 wherein the vertical displacement is defined by a height difference from the fluid level to the reference.

4. The device of claim 1 further comprising a fluid vessel, the fluid vessel coupled to the reference, the sensor in communication with the fluid vessel and configured for detecting a pressure change therein.

5. The device of claim 1 further comprising a measured fluid vessel and a reference fluid vessel, the sensor configured to detect an inequality in pressure between the measured fluid vessel and the reference fluid vessel.

6. The device of claim 5 further comprising a measurement circuit connected to the sensor, the sensor receiving a measured pressure from the measured fluid vessel and a reference pressure from the reference fluid vessel,
the linear actuator configured for adjusting a height of the fluid management drain for attaining an equality of the reference pressure and the measured pressure.

7. The device of claim 2 wherein the sensor is a differential pressure sensor and the circuit is configured to energize the linear actuator until a pressure equality is detected in the pressure defined by the fluid level in response to the vertical displacement.

8. The device of claim 4 further comprising an attachment to a therapeutic equipment stand, the attachment suspending the linear actuator and the receptacle at an elevation above the reference, the elevation inducing the pressure change based on gravitational forces on the fluid vessel.

9. The device of claim 2 wherein the circuit is configured for an agitation of the fluid from the physiological process by a cyclic variation of a vertical position of the fluid management drain for iterative fluid level variation.

10. The device of claim 1 wherein the fluid level is based on a patient cerebrospinal fluid (CSF) accumulation.

11. The device of claim 10 wherein the fluid level emanates from an intercranial cannula having fenestrations for CSF flow.

12. The device of claim 2 wherein the circuit includes a processor and a memory, the memory including instructions for a control loop for computing the height differential, the linear actuator responsive to the control loop based on the computed height differential.

13. A method for maintaining cerebrospinal fluid (CSF) levels in a CSF drain of an intercranial surgical patient, comprising:
locating a reference fluid vessel in association with a vertical location defined by an external ventricular drains (EVD) device for maintaining a pressure in the CSF drain;
locating a measured fluid vessel in association with a patient drain position defined by a CSF drain cannula;
attaching a drain pressure management device including a linear actuator to therapeutic equipment stand;
engaging the external ventricular drains (EVD) device to the linear actuator for vertical adjustment above the EVD drain location; and
activating the linear actuator from a control circuit for maintaining a vertical alignment with the EVD device with the CSF drain cannula based on a pressure equality between the reference fluid vessel and the measured fluid vessel.

14. The method of claim 13 further comprising:
measuring a pressure in the reference fluid vessel;
measuring a pressure in the measured fluid vessel;
detecting a change in the pressure in the measured fluid vessel based on patient movement of the CSF drain cannula; and
realigning EVD device with the CSF drain cannula based on a detecting a subsequent a pressure equality between the reference fluid vessel and the measured fluid vessel.

15. The method of claim 13 further comprising measuring the equality with a differential pressure sensor.

16. A device for maintaining pressure in a surgical drain, comprising:
a reference to a fluid level resulting from a physiological process;
a sensor responsive to a vertical displacement from the reference;
a linear actuator; and
an attachment between the linear actuator and a fluid management drain configured for receiving excess fluid generated by the physiological process based on a pressure defined by the fluid level,
the linear actuator responsive to the sensor for maintaining the vertical displacement relative to the reference.
